(19) United States
(12) Patent Application Publication
(10) Pub. No.: US 2003/0149273 A1
(43) Pub. Date: Aug. 7, 2003

(54) Complexes of N-heterocyclic carbenes and the use thereof

(76) Inventors: Hans-Christian Militzer, Odenthal (DE); Ulrich Scholz, Mulheim (DE); Wolfgang A. Herrmann, Freising (DE); Christian Gstottmayr, Basel (DE)

Correspondence Address: BAYER CHEMICALS CORPORATION, 100 BAYER ROAD, PITTSBURGH, PA, 15205, US

(21) Appl. No.: 10/279,895

(22) Filed: Oct. 24, 2002

(30) Foreign Application Priority Data
Oct. 26, 2001 (DE) 10152989.9

(51) Int. Cl. C07F 15/00; C07D 207/46

(52) U.S. Cl. 548/101; 548/335.1; 548/577; 585/422

(57) ABSTRACT

The present invention relates to a process for preparing polyaryl compounds by coupling aryl halides or aryl sulphonates and reactive aryl compounds in the presence of novel transition metal complexes of N-heterocyclic carbenes which, just like the N-heterocyclic carbenes themselves and the salts from which they are derived, form part of the invention.

The present invention relates to complexes of N-heterocyclic carbenes, to the use thereof and to the precursors of such complexes.

Polyaryl compounds, especially substituted biphenyls, are of great importance as fine chemicals and intermediates for producing medicaments and agrochemicals.

Polyaryl compounds can be synthesized for example by the coupling, with transition metal catalysis, of activated bromo- or iodoaromatic compounds with aryl-grignard, aryl zinc, trialkylaryl compounds or arylboronic acids, where appropriate in the presence of a base (see, for example, Suzuki, A. J., J. Organomet. Chem., 576, 1999, 329-340). The disadvantage of the synthesis, described in the reference, using arylboronic acids is, however, the use of the costly bromo- and iodoaromatic compounds, and the reaction conditions which are in some cases very drastic.

There have already been tests, as catalysts for preparing polyaryl compounds from the lower-cost chloroaromatic compounds, of transition metal complexes of N-heterocyclic carbenes. Zhang and Trudell for example developed (Tetrahedron Letters, 41, 2000, pp. 595-598) a process in which bisimidazol-2-ylidene palladium complexes are employed as catalysts and are generated in situ from palladium acetate and bisimidazolium salts. However, the preparation of such bisimidazolium salts is very elaborate and the process is therefore unsuitable for industrial use.

A similar process of Nolan (J. Org. Chem., 1999, 64, 3804-3805) is based on catalysis by systems, generated in situ, of palladium dibenzylidene acetone [Pd₂(dba)₃] and the monoimidazolium salt 1,3-bis(2,4,6-trimethylphenyl)imida-zolium hydrochloride. However, the reactivity of these systems depends very greatly on the choice of the base employed and is, as was shown by Böhm et al. (J. Organomet. Chem., 595, 2000, pp. 186-190), not applicable to other monoimidazolium salts. Thus, for example, the system palladium dibenzylidene acetone [Pd₂(dba)₃] and 1,3-bis(tert-butyl)imidazolium tetrafluoroborate completely lacks catalytic activity.

The same authors therefore also employed as catalysts defined palladium-imidazol-2-ylidene complexes of the type [Pd(imidazol-2-ylidene)₂], which can be isolated. The isolated complexes which were used showed, however, only moderate conversion rates at 80° C. No reaction was observed at 40° C.

There was thus a need to develop catalysts which are advantageously suitable for a process for preparing polyaryl compounds, in particular starting from aryl chlorides, under mild reaction conditions.

A process for preparing polyaryl compounds has now been found and is characterized in that

aryl halides or aryl sulphonates are reacted together

with reactive aryl compounds

where appropriate in the presence of base and

where appropriate in the presence of solvent and

in the presence of a catalyst which comprises at least one complex of a transition metal selected from the group of nickel, palladium or platinum, which in turn comprises as ligand at least one N-heterocyclic carbene of the general formula (I)

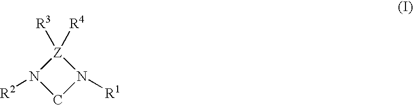

in which

Z is a 1,2-ethanediyl or 1,2-ethenediyl radical, preferably a 1,2-ethenediyl radical, and

R¹ and R² are each, independently of one another, radicals of the general formula (II)

CR⁵R⁶R⁷ (II)

in which

a) CR⁵R⁶R⁷ as a whole is a substituted or unsubstituted carbocyclic or heterocyclic radical or a substituted or unsubstituted carbopolycyclic or heteropolycyclic radical, or

b) the radicals R⁵, R⁶ and R⁷ are each hydrogen or an organic radical,

with the proviso both for a) and for b) that, of the three atoms of R⁵, R⁶ and R⁷ which are bonded to the carbon atom C, either

all three are, in each case independently of one another, secondary, tertiary or quaternary carbon atoms, or

two are, in each case independently of one another, secondary, tertiary or quaternary carbon atoms and, in the case where both of these two atoms are secondary, at least one thereof is bonded to a total of at least two tertiary or quaternary carbon atoms, and

R³ and R⁴ are each, independently of one another, hydrogen, C₆-C₁₂-aryl such as, for example, phenyl, C₆-C₁₂-arylalkyl such as, for example, benzyl or C₁-C₈-alkyl such as, for example, methyl, ethyl or isospropyl.

Examples of preferred reactive aryl compounds are trialkyltinaryl compounds, arylboronic acids, arylzinc and arylmagnesium compounds.

In the process according to the invention for preparing polyaryl compounds there is preferably use of aryl halides or aryl sulphonates of the general formula (III) and aryl compounds of the general formulae (IVa, b, c and d), of which aryl compounds of the formula (IVa) are further preferred.

In the formulae

Ar¹—Y (III)

Ar²—B(OH)₂ (IVa)

Ar²—Sn(C₁-C₆-alkyl)₃ (IVb)

Ar²—ZnX (IVc)

Ar²—MgX (IVd)

Ar¹ and Ar² are each, independently of one another, a substituted or unsubstituted aromatic radical or a substituted or unsubstituted heteroaromatic radical and

Y is chlorine, bromine, iodine or a sulphonate and

X is chlorine, bromine or iodine.

Examples of sulphonates are trifluoromethanesulphonate, pentafluoroethane-sulphonate or nonafluorobutanesulphonate.

Y is particularly preferably chlorine or bromine and very particularly preferably chlorine.

Substituted or unsubstituted aromatic radicals are preferably carbocyclic aromatic radicals having 6 to 24 carbon atoms in the framework, such as, for example, phenyl, naphthyl, biphenyl, binaphthyl or anthracenyl, which may furthermore be substituted by up to five identical or different substituents on each ring.

Substituted or unsubstituted heteroaromatic radicals are preferably heteroaromatic radicals having 5 to 24 carbon atoms in the framework, in which zero, one, two or three carbon atoms in the framework of each ring, but at least one carbon atom in the whole framework of the molecule, may be replaced by heteroatoms selected from the group of nitrogen, sulphur or oxygen, such as, for example, pyrrolyl, pyrazolyl, pyrimidinyl, pyridinyl, oxazolyl, thiophen-yl, furanyl, indolyl, triazolyl, thiazolyl, dibenzofuranyl, dibenzothiophenyl or quinolinyl and which may furthermore be substituted by up to five identical or different substituents on each ring.

Substituents for carbocyclic aromatic or heteroaromatic radicals may be selected, for example, from the group of OH, iodine, bromine, chlorine, fluorine, nitro, cyano, free or protected formyl, C₁-C₁₂-alkyl, C₂-C₁₂-alkenyl, C₆-C₁₂-aryl, C₇-C₁₃-arylalkyl, C₁-C₈-hydroxyalkyl, C₁-C₈-hydroxyalkoxy, C₁-C₈-hydroxyalkylamino, —PO—[(C₁-C₈)-alkyl]₂, —PO—[(C₆-C₁₂)-aryl]₂, tri(C₁-C₆-alkyl)siloxyl or radicals of the general formula (V)

A-B-D-E (V)

in which, independently of one another,

A is absent, is a C₁-C₈-alkylene radical or a C₂-C₈-alkenylene radical, and

B is absent or is oxygen, sulphur or NR⁸,

where R⁸ is hydrogen, C₁-C₈-alkyl, C₇-C₁₀-arylalkyl or C₆-C₁₀-aryl, and

D is a carbonyl group, and

E is R⁹, OR⁹ or N(R¹⁰)₂,

where

R⁹ is hydrogen, C₁-C₈-alkyl, C₇-C₁₀-arylalkyl, C₁-C₈-hydroxyalkyl, C₁-C₈-haloalkyl or C₆-C₁₀-aryl and

R¹⁰ is in each case independently, hydrogen, C₁-C₈-alkyl, C₁-C₈-hydroxyalkyl, C₇-C₁₀-arylalkyl or C₆-C₁₀-aryl or N(R¹⁰)₂ as a whole as a cyclic amino radical,

or radicals of the general formulae (VIa-e)

A-E (VIa)

A-SO₂-E (VIb)

A-B—SO₂R⁹ (VIc)

A-SO₃X (VId)

A-COX (VIe)

in which A, B, E and R⁹ have the meaning indicated above, and X is OM, where M may be an alkali metal ion, a half equivalent of an alkaline earth metal ion, an ammonium ion or an organic ammonium ion. M is preferably lithium, sodium, potassium, ammonium or organic ammonium ions of the general formula (VII)

[NHₙ(C₁-C₁₂-alkyl)ₘ(C₂-C₆-hydroxyalkyl)ₚ(C₇-C₁₂-arylalkyl)_{q}(C₆-C₁₀-aryl)ᵣ]⁺ (VII),

in which (n+m+p+q+r)=4.

M may also be hydrogen. However, in this case, the acidic groups are then present in the reaction medium in the form of the salts of the base employed.

Alkyl or alkylene means in all contexts of the invention, in each case independently, a straight-chain, cyclic, branched or unbranched alkyl or alkylene radical which may optionally be further substituted by alkoxy groups. The same applies to the alkyl moiety of an arylalkyl radical.

The general term aryl as substituent means in all contexts of the invention not only carbocyclic radicals but also heteroaromatic radicals in which zero, one, two or three carbon atoms in the framework of each ring, but at least one carbon atom in the whole framework of the radical, is replaced by heteroatoms selected from the group of nitrogen, sulphur or oxygen.

Alkoxy means in all contexts of the invention, in each case independently, a straight-chain, cyclic or branched or unbranched alkoxy radical.

Haloalkyl and haloalkoxy mean in all contexts of the invention, in each case independently, straight-chain, cyclic, branched or unbranched alkyl radicals and alkoxy radicals which may be substituted by one, more than one or completely by fluorine or chlorine atoms. It is furthermore possible for these radicals to be substituted further by alkoxy radicals.

Ar¹ and Ar² are particularly preferably, in each case independently of one another, the carbocyclic aryl radicals phenyl, naphthyl, biphenyl, binaphthyl or anthracenyl, or heteroaryl radicals selected from the group of pyrrolyl, pyrimidinyl, pyridinyl, oxazolyl, thiophen-yl, furanyl, indolyl or quinolinyl, each of which may be further substituted by zero, one, two or three substituents selected from the group of OH, iodine, bromine, chlorine, fluorine, nitro, cyano, free or protected formyl, C₁-C₄-alkyl, benzyl, C₁-C₄-hydroxyalkyl, —PO—[(C₁-C₈)-alkyl]₂, —PO—[(C₆-C₁₂-aryl]₂, or radicals of the general formula (V) in which, independently of one another,

A is absent and

B is absent or is NR⁸,

where

R⁸ is hydrogen or C₁-C₄-alkyl, and

D is a carbonyl group, and

E is R⁹, OR⁹ or N(R¹⁰)₂,

where R⁹ is hydrogen, C₁-C₈-alkyl or C₆-C₁₀-aryl and

R¹⁰ is, in each case independently, hydrogen, C₁-C₈-alkyl or N(R¹⁰)₂ as a whole is a pyrrolidinyl or morpholinyl radical,

or radicals of the general formulae (VId) or (VIe) in which

A has the meaning indicated above, and X is ONa or OK.

Ar¹ and Ar² are particularly preferably, in each case independently of one another, phenyl, pyrrolyl, pyrimidinyl, pyridinyl radicals which are further substituted by zero, one or two substituents selected from the group of fluorine, nitro, cyano, formyl, methyl, ethyl, methoxy, trifluoromethyl, amino, dimethylamino, aminoacetyl, acetyl, COONa or SO₃Na.

The compounds of the general formula (III) which are very particularly preferably employed for the process according to the invention are 4-chlorotoluene, 2-chlorotoluene, 1-chloro-4-trifluoromethylbenzene, 1-chloro-4-methoxybenzene and 1-chloro-4-acetylbenzene.

Compounds of the general formula (IVa) are very particularly preferably employed for the process according to the invention, particularly preferably phenylboronic acid, 2-methylphenylboronic acid and 3-methoxyphenylboronic acid.

The aryl halides or aryl sulphonates which can be employed in the reaction, and the reactive aryl compounds are either commercially available or can be prepared by literature methods or in analogy thereto.

The molar ratio of aryl halide or aryl sulphonate to reactive aryl compound employed can be, for example, 0.01:1 to 100:1, and a molar ratio of 0.5:1 to 5:1 is preferred, particularly preferably 0.8:1 to 1:1.5.

In a preferred embodiment of the process according to the invention, arylboronic acids are employed as reactive aryl compounds, and bases are employed, such as, for example:

nitrogen bases such as, for example, pyridine or amines such as diethylamine or triethylamine,

alkali metal or alkaline earth metal bicarbonates, carbonates, alcoholates, carboxylates or fluorides or organic ammonium fluorides or mixtures of bases.

The bicarbonates, carbonates, (2 base equivalents) methanolates, ethanolates, isopropoxides, tert-butanolates and acetates of lithium, sodium, potassium and caesium, and caesium fluoride, are preferably employed.

Caesium fluoride and carbonate are particularly preferred.

Caesium fluoride is very particularly preferred.

The molar ratio of base equivalents to reactable aryl halide or aryl sulphonate can be, for example, 0.5:1 to 100: 1, and a molar ratio of 1:1 to 5:1 is preferred, particularly preferably 1:1 to 1.5:1.

Reactable means in this connection that proportion of aryl halide or aryl sulphonate for which one equivalent of arylboronic acid is employed in the reaction.

The process according to the invention is, where appropriate, carried out in the presence of one or more aprotic solvents. These are preferably:

cyclic or acyclic ethers such as, for example, 1,4-dioxane, tetrahydrofuran, diethyl ether, methyl tert-butyl ether or di-n-butyl ether, aromatic hydrocarbons such as, for example, toluene, o-xylene, m-xylene or p-xylene, dipolar aprotic compounds such as, for example, dimethylformamide or N-methylpyrrolidone, dimethyl acetatamide, dimethyl sulphoxide or mixtures of such solvents, which may also contain water. In the case of liquid aryl halides or aryl sulphonates, the latter can also be employed as solvent itself in excess.

1,4-Dioxane is particularly preferred.

The amount of aprotic solvent employed where appropriate can be, for example, 50 ml to 5.000 ml, preferably 500 to 3.000 ml, per mole of the aryl halide or aryl sulphonate employed.

Preferred N-heterocyclic carbenes are those of the general formula (I) in which

Z is a 1,2-ethenediyl radical and the two radicals R¹ and R² are identical and are radicals of the general formula (II), and in which CR⁵R⁶R⁷

a) is in each case as a whole a substituted or unsubstituted C₅-C₂₀ carbocyclic radical or a substituted or unsubstituted C₆-C₂₄ carbopolycyclic radical, or

b) R⁵, R⁶ and R⁷ can, in each case independently, be an organic radical selected from the group of C₁-C₂₀-alkyl, C₁-C₁₂-haloalkyl, C₇-C₂₀-arylalkyl, C₅-C₁₈-aryl or radicals of the general formula (V)

with the abovementioned proviso applying both for a) and for b).

Particularly preferred N-heterocyclic carbenes of the general formula (I) are those in which Z is a 1,2-ethenediyl radical and the two radicals R¹ and R² are identical and are radicals of the general formula (II), and in which CR⁵R⁶R⁷

a) in each case as a whole is in each case one of the 8 isomeric menthyl radicals or a substituted or unsubstituted adamantyl radical, or

b) R⁵, R⁶ and R⁷ are each ethyl, n-propyl, isopropyl, n-butyl, n-pentyl, n-hexyl, cyclohexyl, n-octyl, isooctyl, trifluoromethyl, benzyl, phenyl, 1-naphthyl or 2-naphthyl.

Examples of possible substituents on the adamantyl radical are:

oxo, C₁-C₆-alkoxy, fluorine, (C₁-C₄)-acyloxy, cyano, unbranched or branched straight-chain or cyclic C₁-C₆-alkyl such as, for example, methyl, ethyl, isopropyl, cyclopentyl, cyclohexyl, (C₁-C₄)-acylamino, C₁-C₆-haloalkyl such as, for example, trifluoromethyl, (C₁-C₆)-alkoxycarbonyl, unsubstituted or substituted phenyl such as, for example, phenyl, nitrophenyl, p-, o-, m-tolyl, p-, o-, m-anisyl.

Very particularly preferred N-heterocyclic carbenes of the general formula (I) are 1,3-di-(1R, 2S, 5R-(−)menthylimidazolin-2-ylidene, 1,3-di-(1S, 2R, 5S —)-(+)methylimidazolin-2-ylidene and 1,3-diadamantylimidazolin-2-ylidene.

The most preferred N-heterocyclic carbene ligand of the general formula (I) is 1,3-diadamantylimidazolin-2-ylidene.

The catalysts preferably employed for the process according to the invention are nickel or palladium complexes in the formal oxidation state of zero, which comprise per metal atom at least one N-heterocyclic carbene of the general formula (I) in which Z, R¹, R², R³ and R⁴ each have the stated meaning, independently of further N-heterocyclic carbenes of the general formula (I) which are present where appropriate.

Particularly preferred catalysts are palladium complexes in the formal oxidation state of zero, which comprise per metal atom at least one N-heterocyclic carbene of the general formula (I) in which Z, R¹, R², R³ and R⁴ each have the stated meaning, independently of further N-heterocyclic carbenes of the general formula (I) which are present where appropriate.

Very particularly preferred complexes are palladium complexes of the general formula (VIII)

[Pd(L)₂] (VIII)

in which the two ligands L are each, independently of one another, N-heterocyclic carbenes of the general formula (I) in which Z, R¹, R², R³ and R⁴ each, independently of one another, have the meaning mentioned there.

The two ligands L in formula (II) are preferably identical.

The most preferably employed catalysts are the complexes [bis(1,3-diadamantyl-imidazol-2-ylidene)palladium], [bis(1,3-di-(−)-menthylimidazol-2-ylidene)palla-dium] and [bis(1,3-di-(−)-menthylimidazol-2-ylidene)palladium], of which [bis(1,3-diadamantylimidazol-2-ylidene)palladium] is even more preferred.

It may be pointed out at this juncture that the invention encompasses any combinations of all the preferred ranges.

The complexes employed as catalysts can be prepared, for example, by ligand substitution reactions on a suitable precursor complex.

Palladium complexes of the general formula (VIII) can be prepared directly, for example in analogy to Cloke (J. Organomet. Chem., 2001, 617-618, 635-639), by reacting the N-heterocyclic carbenes of the general formula (I) with allylpalladium chloridedimer and sodium dimethyl malonate.

It is furthermore possible for example for palladium complexes of the general formula (VIII) also to be prepared in an advantageous way by reacting palladium complexes of the general formula (IX)

[Pd(P)₂] (IX)

in which P is a monodentate phosphane ligand, with the N-heterocyclic carbenes of the general formula (I) in the presence of solvent. [Bis(tri-tert-butylphosphane)-palladium ] is preferably employed as palladium complex of the general formula (IX) in this case.

Examples of solvents suitable for the reaction are ethers such as, for example, tetrahydrofuran, aliphatic or aromatic hydrocarbons such as, for example, pentene, n-hexane, cyclohexane, toluene.

Hexane is particularly preferably employed as solvent in this case.

The temperature can be, for example, between −20° C. and 80C, and 10 to 50° C. are preferred, and room temperature is particularly preferred.

It is possible by the described process, which is likewise encompassed by the invention, to obtain palladium complexes of the general formula (VIII) in high yields.

If N-heterocyclic carbenes of the general formula (I) are required for syntheses of complexes according to the invention, these can take place in the manner known per se by deprotonation from the analogous salts of the general formula (X)

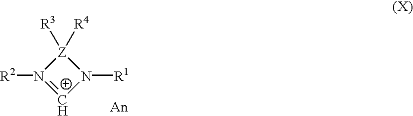

in which Z, R¹, R², R³ and R⁴ have the meanings mentioned for formula (I), and in which An is the anion of an acid.

Formula (X) represents the possible tautomeric compounds which are likewise encompassed by the scope of the invention.

An is preferably an anion of an acid which has a pKa of 3 or less. An is particularly preferably hydrogen sulphate, chloride, bromide, iodide, tetrafluoroborate, hexafluorophosphate or a half equivalent of sulphate.

An is very particularly preferably chloride.

The deprotonation is moreover preferably effected by alkali metal hydrides such as, for example, sodium hydride in a mixture of an ether such as, for example, THF and liquid ammonia at temperatures between −35 and −80° C.

The salts according to the invention of the general formula (X) can be prepared for example by stepwise alkylation of compounds of the general formula (XI)

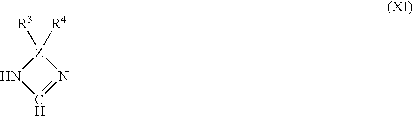

in which

Z, R³ and R⁴ have the meaning indicated for formula (I).

If the radicals R¹ and R² in the salts of the general formula (X) are identical, and if Z is a 1,2-ethenediyl radical, the preparation preferably takes place by reacting amines of the general formula (XII)

H₂N—R¹ (XII)

in which R¹ has the meaning mentioned for the general formula (I),

with vicinal dicarbonyl compounds

of the general formula (XIII)

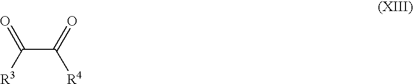

and formaldehyde

in the presence of an acid of the general formula (XIV)

H-An (XIV).

in which An has the meaning indicated for the general formula (X).

The salts of the general formula (X) likewise form part of the invention and can be employed either directly, for example by the method of Cloke (loc.cit.), or after previous deprotonation for preparing the catalysts and complexes according to the invention.

The compounds of the general formula (X) in which Z is a 1,2-ethanediyl radical can be prepared for example as described by or in analogy to J. F. Hartwig, Org. Lett. 2000, 2, 10, p. 1423.

The individual stages which may lead to the preparation of the catalysts according to the invention may be represented diagrammatically by the example of [bis(1,3-diadamantylimidazol-2-ylidene)palladium]:

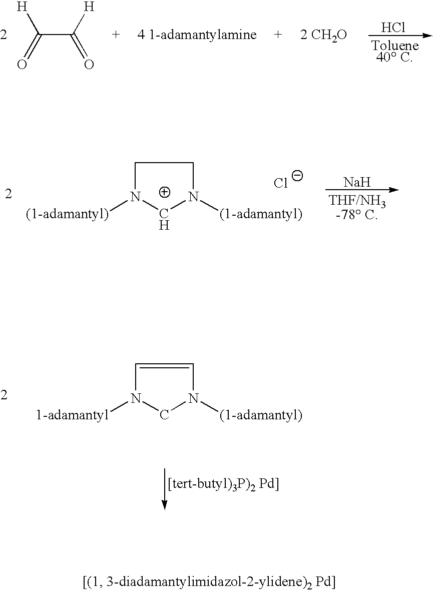

The process according to the invention for preparing polyaryl compounds can be carried out for example at a reaction temperature of 0 to 100° C., preferably 20 to 80° C. Room temperature is very particularly preferred.

The reaction may take, for example, 5 minutes to 168 h, preferably 20 min to 25 h.

The reaction can, for example, be carried out under 0.2 to 100 bar, preferably atmospheric pressure.

The reaction is preferably carried out under protective gas and with substantial exclusion of oxygen and moisture. Examples of suitable protective gases are nitrogen and noble gases such as, for example, argon or mixtures thereof.

In a preferred embodiment, the arylboronic acid of the general formula (IVa), the complex of the general formula (VIII) and the base are introduced where appropriate into a solvent under a protective gas atmosphere, and then the aryl halide or the aryl sulphonate is added, where appropriate dissolved in a solvent, and the mixture is stirred at a temperature between 20 and 80° C. After the reaction is complete (detection for example by GC/MS), water is added to the reaction mixture, and the precipitated palladium black is removed by filtration or centrifugation and can subsequently be recycled. The product can be obtained in a manner known per se, for example by evaporation of solvent, and further purified where appropriate furthermore for example by distillation, sublimation, recrystallization or reprecipitation.

In a further preferred embodiment, the aryl halide or the aryl sulphonate of the general formula (III), the arylboronic acid of the general formula (IVa), the complex of the general formula (VIII) and the base are mixed under a protective gas atmosphere, then solvent is added, and the mixture is stirred at a temperature between 20 and 80° C. until the conversion exceeds 95%.

Polyaryl compounds of the general formula (XV)

Ar¹—Ar² (XV)

in which Ar¹ and Ar² have the meanings mentioned for the general formulae (III) and (IVa to d) are obtained in an advantageous manner in a process according to the invention.

The polyaryl compounds prepared in the manner according to the invention are particularly suitable for preparing medicaments, agrochemicals and polymers, especially conducting polymers.

The particular advantage of the present invention is that the provision of novel salts and the N-heterocyclic carbenes derived therefrom and complexes thereof makes novel catalysts available, which make it possible in a superior manner to carry out Suzuki coupling in particular of aryl chlorides with reactive aryl compounds even at room temperature with very good yields and previously unachieved activities.

### EXAMPLES

### Example 1

Preparation of bis(1,3-diadamantyl-2-ylidene)palladium(0)

Bis-tri-tert.-butylphosphane)palladium(0) (1000 mg, 1.81 mmol) was dissolved in 30 mL of n-hexane. A solution of 1,3-diadamantylimidazol-2-ylidene (1400 mg, 4.16 mmol) in 30 mL of n-hexane was added. The mixture was stirred at room temperature for 24 to 48 h, during which a yellow solid precipitated. Filtration and drying in vacuo resulted in a pale yellow solid. The X-ray structural analysis was carried out after recrystallization from diethyl ether.

Yield: 1 177 mg, 1.51 mmol, 83% of theory.

### Example 2

Alternative Preparation via Phosphane-Free Palladium Precursor

1,3-Diadamantylimidazol-2-ylidene (222.1 mg, 0.66 mmol) allylpalladium(II) chloride dimer (60.4 mg, 0.165 mmol) and sodium dimethyl malonate (50.9 mg, 0.33 mmol) are dissolved in 20 mL of toluene under a nitrogen atmosphere. The mixture is heated in a Schlenk tube at 90° C. for 16 h. Then small amounts of palladium black and produced NaCl are removed by filtration, the filtrate is concentrated to half the volume, and the product is precipitated at −78° C. Yield: 102 mg, 0.13 mmol, 40% of theory.

Melting point>285° C. (decomposition); ¹H NMR (400 MHz, d8-toluene, 25° C.): δ=1.64 (m, 24 H, CH₂C₁₀H₁₅), 180 (m, 12 H, CHC₁₀H₁₅), 2.11 (m, 24 H, CH₂C₁₀H₁₅), 6.69 (s, 4 H, NCHCHN); ¹³C{¹H}NMR (100.5 MHz, d8-toluene, 25° C.); δ=31.0, 36.8, 44.0 (C₁₀H₁₅), 57.2 (iso-C C₁₀H₁₅), 112.7 (NCHCHN), 191.8 (NCN); Cl-MS; m/z (%): 778 (3) [M⁺]336 (100) [NHC⁺], 281 (33), 207 (27), 203 (40); C₄₆H₆₄N₄Pd (779.50): calculated C 70.88, H 8,22, N 7.19; found C 70.80, H 8,24, N 7.22.

### Example 3

Coupling of Aryl Halides with Arylboronic Acids: General Method

Bis(1,3-diadamantyl-2-ylidene)palladium(0) (23.4 mg, 0.03 mmol), CsF (303.8 mg, 2 mmol), the arylboronic acid (1.5 mmol) and the aryl chloride (1 mmol) were introduced under a nitrogen atmosphere into a Schlenk tube. After addition of 3 mL of 1,4-dioxane, the mixture was stirred at room temperature. The reaction was stopped after the stated time by adding a few drops of water, and the palladium black was removed by filtration. The yield was determined by GC/MS analysis with diethylene glycol di-n-butyl ether as internal standard.

The results of the catalyses are shown in Table 1.

TABLE 1

| | | | | Tempera- | Conver- |
|---|---|---|---|---|---|
| | Aryl-X | Aryl-B(OH)₂ | Time | ture | sion |
| a) | p-Chloro- | Phenylboronic acid | 6 h | RT | >95% |
| | toluene | | 20 min | 80° C. | >95% |
| b) | p-Chloro- | 3-Methoxyphenyl- | 25 h | RT | 80% |
| | toluene | boronic acid | 2 h | 80° C. | 88% |
| c) | 1-Chloro-4-tri- | Phenylboronic acid | 2 h | RT | 95% |
| | fluoromethyl- | | | | |
| | benzene | | | | |
| d) | 1-Chloro-4-tri- | 3-Methoxyphenyl- | 25 h | RT | 73% |
| | fluoromethyl- | boronic acid | 2 h | | >95% |
| | benzene | | | | |
| e) | 4-Acetyl-1- | 3-Methoxyphenyl- | 25 h | RT | 95% |
| | chlorobenzene | boronic acid | | | |
| f) | 1-Chloro-4- | Phenylboronic acid | 6 h | RT | >95% |
| | methoxy- | | | | |
| | benzene | | | | |

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Process for preparing polyaryl compounds, characterized in that
aryl halides or aryl sulphonates are reacted together
with reactive aryl compounds
in the presence of a catalyst which comprises at least one complex of a transition metal selected from the group of nickel, palladium or platinum, which in turn comprises at least one N-heterocyclic carbene of the general formula (I) 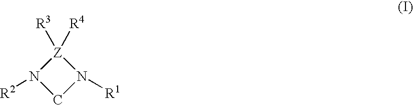
in which
Z is a 1,2-ethanediyl or 1,2-ethendiyl radical, and
R¹ and R² are each, independently of one another, radicals of the general formula (II)
CR⁵R⁶R⁷ (II)
in which
a) CR⁵R⁶R⁷ as a whole is a substituted or unsubstituted carbocyclic or heterocyclic radical or a substituted or unsubstituted carbopolycyclic or heteropolycyclic radical, or
the radicals R⁵, R⁶ and R⁷ are each hydrogen or an organic radical,
with the proviso both for a) and for b) that, of the three atoms of R⁵, R⁶ and R⁷ which are bonded to the carbon atom C, either
all three are, in each case independently of one another, secondary, tertiary or quaternary carbon atoms, or
two are, in each case independently of one another, secondary, tertiary or quaternary carbon atoms and, in the case where both of these two atoms are secondary, at least one thereof is bonded to a total of at least two tertiary or quaternary carbon atoms, and
R³ is hydrogen, methyl, benzyl and
R⁴ is hydrogen, C₁-C₈-alkyl, benzyl or phenyl.

2. Process according to claim 1, characterized in that it is carried out in the presence of base.

3. Process according to claim 2, characterized in that bicarbonates, carbonates, methanolates, ethanolates, isopropoxides, tert-butanolates and acetates of lithium, sodium, potassium and caesium, and caesium fluoride, are employed as base.

4. Process according to claim 2, characterized in that caesium fluoride is employed as base.

5. Process according to claim 1, characterized in that it is carried out in the presence of solvent.

6. Process according to claim 1, characterized in that the aryl halides or aryl sulphonates employed are those of the general formula (III)
Ar¹—Y (III)
in which
Ar¹ is a substituted or unsubstituted aromatic radical or a substituted or unsubstituted heteroaromatic radical and
Y is chlorine, bromine, iodine or a sulphonate.

7. Process according to claim 1, characterized in that the aryl halides or aryl sulphonates employed are those of the general formula (III) in which Ar¹ is a substituted or unsubstituted aromatic radical or a substituted or unsubstituted heteroaromatic radical and Y is chlorine.

8. Process according to claim 1, characterized in that the reactive aryl compounds employed are those of the general formula (IVa, b, c and d)
Ar²—B(OH)₂ (IVa)
Ar²—Sn(C₁-C₆-alkyl)₃ (IVb)
Ar²—ZnX (IVc)
Ar²—MgX (IVd)
in which
Ar2 is a substituted or unsubstituted aromatic radical or a substituted or unsubstituted heteroaromatic radical, and X is chlorine, bromine or iodine.

9. Process according to claim 1, characterized in that palladium complexes of the general formula (VIII)
[Pd(L)₂] (VIII)
in which the two ligands L are identical and are N-heterocyclic carbenes of the general formula (I) in which the radicals Z, R¹, R², R³ and R⁴ have the meaning specified in claim 1, are employed as catalyst.

10. Process according to claim 1, characterized in that palladium complexes of the general formula (VIII) in which the two ligands L are identical and are N-heterocyclic carbenes of the general formula (I) in which Z is a 1,2-ethylenediyl radical and the radicals R¹ and R² are identical and have the meaning specified in claim 1, and R³ and R⁴ are each hydrogen, are employed as catalyst.

11. Process according to claim 1, characterized in that the complex [bis(1,3-diadamantylimidazol-2-ylidene)palladium] is employed as catalyst.

12. Process according to claim 1, characterized in that the reaction temperature is 15 to 40° C.

13. Use of polyaryl compounds which have been prepared by a process according to claim 1 in a process for producing medicaments or agrochemicals.

14. Use of polyaryl compounds which have been prepared by a process according to claim 1 as medicaments or agrochemicals.

15. N-Heterocyclic carbenes of the general formula (I) 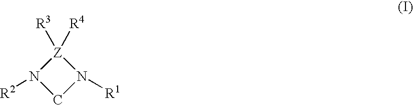
in which
Z is a 1,2-ethanediyl or a 1,2-ethylenediyl radical and
R¹ and R² are each, independently of one another, radicals of the general formula (II)
CR⁵R⁶R⁷ (II)
in which
a) CR⁵R⁶R⁷ as a whole is a substituted or unsubstituted carbocyclic or heterocyclic radical or a substituted or unsubstituted carbopolycyclic or heteropolycyclic radical, or
b) the radicals R⁵, R⁶ and R⁷ are each hydrogen or an organic radical,
with the proviso both for a) and for b) that, of the three atoms of R⁵, R⁶ and R⁷ which are bonded to the carbon atom C, either
all three are, in each case independently of one another, secondary, tertiary or quaternary carbon atoms, or
two are, in each case independently of one another, secondary, tertiary or quaternary carbon atoms and, in the case where both of these two atoms are secondary, at least one thereof is bonded to a total of at least two tertiary or quaternary carbon atoms, and
R³ and R⁴ are each independently hydrogen, C₆-C₁₂-aryl, C₆-C₁₂-arylalkyl or C₁-C₈-alkyl.

16. 1,3-Diadamantylimidazol-2-ylidene.

17. Salts of the general formula (X) 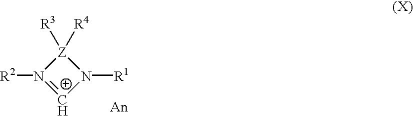
in which
Z is a 1,2 ethanediyl or a 1,2-ethenediyl radical and
R¹ and R² are each, independently of one another, radicals of the general formula (II)
CR⁵R⁶R⁷ (II)
in which
a) CR⁵R⁶R⁷ as a whole is a substituted or unsubstituted carbocyclic or heterocyclic radical or a substituted or unsubstituted carbopolycyclic or heteropolycyclic radical, or
b) the radicals R⁵, R⁶ and R⁷ are each hydrogen or an organic radical,
with the proviso both for a) and for b) that, of the three atoms of R⁵, R⁶ and R⁷ which are bonded to the carbon atom C, either
all three are, in each case independently of one another, secondary, tertiary or quaternary carbon atoms, or
two are, in each case independently of one another, secondary, tertiary or quaternary carbon atoms and, in the case where both of these two atoms are secondary, at least one thereof is bonded to a total of at least two tertiary or quaternary carbon atoms, and
R³ and R⁴ are each independently hydrogen, C₆-C₁₂-aryl, C₆-C₁₂-arylalkyl or C₁-C₈-alkyl and
An is the anion of an acid.

18. 1,3-Diadamantylimidazolium chloride.

19. Nickel, palladium and platinum complexes which comprise as ligands at least one N-heterocyclic carbene of the general formula (I) 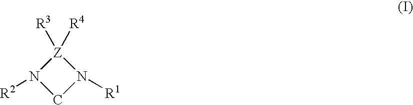
in which
Z is a 1,2-ethanediyl or a 1,2-ethylenediyl radical and
R¹ and R² are each, independently of one another, radicals of the general formula (II)
CR⁵R⁶R⁷ (II
in which
a) CR⁵R⁶R⁷ as a whole is a substituted or unsubstituted carbocyclic or heterocyclic radical or a substituted or unsubstituted carbopolycyclic or heteropolycyclic radical, or
b) the radicals R⁵, R⁶ and R⁷ are each hydrogen or an organic radical,
with the proviso both for a) and for b) that, of the three atoms of R⁵, R⁶ and R⁷ which are bonded to the carbon atom C, either
all three are, in each case independently of one another, secondary, tertiary or quaternary carbon atoms, or
two are, in each case independently of one another, secondary, tertiary or quaternary carbon atoms and, in the case where both of these two atoms are secondary, at least one thereof is bonded to a total of at least two tertiary or quaternary carbon atoms, and
R³ and R⁴ are each independently hydrogen, C₆-C₁₂-aryl, C₆-C₁₂-arylalkyl or C₁-C₈-alkyl.

20. [Bis(1,3-diadamantylimidazol-2-ylidene)palladium].

21. Use of compounds according to claim 15 for preparing transition metal complexes.

22. Use of compounds according to claim 17 for preparing N-heterocyclic carbenes or transition metal complexes thereof.

23. Use of compounds according to claim 19 as catalyst.

24. Process for preparing complexes of the general formula (VIII)
[Pd(L)₂] (VIII)
in which the two ligands L are each, independently of one another, N-heterocyclic carbenes, characterized in that
palladium complexes of the general formula (IX)
[Pd(P)₂] (IX)
in which P is a monodentate phosphane ligand are reacted with an N-heterocyclic carbene in the presence of solvent.
